(19) United States
(12) Patent Application Publication
(10) Pub. No.: US 2021/0315955 A1
(43) Pub. Date: Oct. 14, 2021

(54) COMPOSITIONS AND METHODS FOR TREATMENT OF RESPIRATORY AILMENTS

(71) Applicants: Bert Forman, West Palm Beach, FL (US); Jongkochpor Phinitaksorn, Bangkok (TH)

(72) Inventors: Bert Forman, West Palm Beach, FL (US); Jongkochpor Phinitaksorn, Bangkok (TH)

(21) Appl. No.: 17/226,361

(22) Filed: Apr. 9, 2021

Related U.S. Application Data
(60) Provisional application No. 63/008,106, filed on Apr. 10, 2020.

(51) Int. Cl. A61K 36/428; A61P 11/00; A61K 36/54; A61K 36/61; A61K 36/23; A61K 9/00; A61P 31/00; A61K 31/522; A61K 36/752

(52) U.S. Cl. CPC A61K 36/428; A61P 11/00; A61K 36/54; A61K 36/61; A61K 36/752; A61K 9/0056; A61P 31/00; A61K 31/522; A61K 36/23

(57) ABSTRACT

Compositions and methods for treating respiratory ailments are disclosed.

### BACKGROUND OF THE DISCLOSURE

### Field of the Disclosure

This disclosure relates generally to therapeutic compositions and methods. This disclosure relates particularly to compositions and methods useful for treating respiratory ailments, such as those associated with microbial infection, viral infection, allergies, and/or asthma and others.

### Description of Related Art

Sinusitis (rhinosinusitis) is a condition in which the mucus membranes lining the sinuses associated with the nasal cavity become inflamed and which results in the overproduction sinonasal secretions and post-nasal drip. Sinusitis can be caused by viral infections such as the common cold and influenza, bacterial infections (e.g., Staphylococcus aureus), allergies, and air pollution. Chronic rhinosinusitis is defined as persistent symptomatic inflammation of the nasal and sinus mucosa that last longer than 12 weeks.

Chronic rhinosinusitis (CRS) is one of the most common chronic diseases, with a prevalence as high as 15% of the population in some studies (Suh et al., Proc Am Thorac Soc. 8: 132-40 (2011)). The incidence of CRS, similar to asthma, appears to be increasing, which has significant socioeconomic implications. CRS causes millions of office visits and billions of dollars of health care costs each year (Benninger et al., Otolaryngol Head Neck Surg. 129: S1-S32 (2003)).

The epidemiological association of CRS with asthma is well known (Ozcan et al., Arch Otolaryngol Head Neck Surg. 129: 1006-09 (2003)). Indeed, sinonasal pathology is the most common comorbidity among patients with asthma (Dixon, Curr Opin Pulm Med. 15(1): 19-24 (2009)). Aspiration of sinonasal secretions into the lungs is hypothesized to be potentially related to the pathogenesis of asthma. In addition, more recently, bacterial biofilms (and perhaps multifloral bacterial/fungal biofilms) have been associated pathologically with CRS, which suggests a potential role for CRS biofilms in the pathophysiology of associated respiratory ailments, like asthma (Fastenberg et al., World J Otorhinolaryngol. Head Neck Surg. 2(4): 219-29 (2016)).

Similar to asthma, chronic obstructive pulmonary disease (COPD) is a long-term, progressive lung disease characterized by obstruction of airflow due to inflammation of the bronchi and by excess mucus production. Typically, COPD is the result of exposure to lung irritants, such as cigarette smoke, air pollution, and chemical fumes. However, the relevance of bacterial infections in the exacerbation of COPD is becoming increasingly clear (Moghoofei et al., Infection 48(1): 19-35 (2020)).

Approaches to treating CRS include administration of steroids, saline nasal irrigation, antibiotics, antifungals, antihistamines, decongestants, mucolytics, and leukotriene inhibitors, as well as surgery (Suh et al., Proc Amer Thoracic Soc. 8: 132-40 (2011)). Treatments for asthma often include administration of steroids, beta-2 agonists, methylxanthines, mast cell stabilizers, leukotriene modulators, and immunomodulators. Treatments for COPD are often steroidal bronchodilators. Each of the foregoing treatment regimens has its place; but such treatments can have their drawbacks, such as short-lived effectiveness or complete ineffectiveness, serious side-effects, and/or significant costs.

Based on the foregoing, new compositions and methodologies are needed to provide alternative, safe approaches to treating CRS, asthma, COPD, and related respiratory ailments, as well as tachyphylaxis in association with such treatments.

### SUMMARY OF THE DISCLOSURE

As described in detail below, the disclosure provides methods for treating respiratory ailments as well as compositions therefor. Other aspects, embodiments, and implementations will become apparent to those of ordinary skill in the art by reading the following detailed description, with reference where appropriate to the accompanying drawings.

In a first aspect, the disclosure provides a method of treating chronic rhinosinusitis (CRS) in a subject in need thereof, comprising: a) administering a therapeutically effective amount of a composition to the subject, the composition comprising: i) a therapeutically effective amount of a Trichosanthes fruit extract or one or more active ingredients thereof, and ii) optionally, a second essential oil or active ingredient thereof; and b) reducing one or more symptoms of CRS in the subject.

In one embodiment of the first aspect, the method reduces one or more of nasal and/or respiratory congestion, post-nasal drip, wheezing, sneezing, coughing, shortness of breath, insomnia, mucus production in the nasal cavity and/or respiratory tract and/or lungs. In another embodiment of the first aspect, reduction in one or more symptoms is a result of one or more of decreased biofilm mass or decreased bacterial density in the subject's respiratory tract and/or increased lung capacity. In another embodiment of the first aspect, the method reduces inflammation within the respiratory tract of the subject. In another embodiment of the first aspect, the composition comprises about 0.1 to about 99% of the Trichosanthes fruit extract or an active ingredient thereof. In another embodiment of the first aspect, the active ingredient of Trichosanthes fruit extract comprises one or more of (e)-beta-ocimene, anethole, b-elemen, borneol, cadina-1,4-diene, cadinene isomer, gamma-cadinene, alpha-cadinene, delta-cadinene, camphor, beta-caryophyllene, alpha-cedrene, beta-cedrenea, cedrol crystals, alpha-chamigrene, alpha-copaene, ethyl amyl ketone, eucalyptol, farnesene, alpha-farnesene, trans beta-farnesene, gamma-terpinene, geranoxide, geranyl acetate, d-germacrene, guaiene, alpha-humulene, isoborneol, isobornyl acetate, isolongifolene, levo-juneol, linalool, menthol, menthone, menthyl acetate, methyl benzoate, methyl chavicol, methyl salicylate, muurolene gamma, alpha-muurolol, neryl acetate, nopyl acetate, oleyl alcohol, para cresyl methyl ether, para cymene, alpha-pinene, terpinyl acetate, tetradecane, thujopsadien, thujopsene, thymol, widdrol, and ylangene, and combinations thereof. In one embodiment of the first aspect, the active ingredient of Trichosanthes fruit extract comprises at least 4 or more of (e)-beta-ocimene, anethole, b-elemen, borneol, cadina-1,4-diene, cadinene isomer, gamma-cadinene, alpha-cadinene, delta-cadinene, camphor, beta-caryophyllene, alpha-cedrene, beta-cedrenea, cedrol crystals, alpha-chamigrene, alpha-copaene, ethyl amyl ketone, eucalyptol, farnesene, alpha-farnesene, trans beta-farnesene, gamma-terpinene, geranoxide, geranyl acetate, d-germacrene, guaiene, alpha-humulene, isoborneol, isobornyl acetate, isolongifolene, levo-juneol, linalool, menthol, menthone, menthyl acetate, methyl benzoate, methyl chavicol, methyl salicylate, muurolene gamma, alpha-muurolol, neryl acetate, nopyl acetate, oleyl alcohol, para cresyl methyl ether, para cymene, alpha-pinene, terpinyl acetate, tetradecane, thujopsadien, thujopsene, thymol, widdrol, and ylangene.

In another embodiment of the first aspect, the composition comprises: about 10% to about 20% Trichosanthes fruit extract or an active ingredient thereof. In another embodiment of the first aspect, the composition further comprises: about 15 to about 25% bergamot essential oil or an active ingredient thereof; about 15 to about 25% clove bud oil or an active ingredient thereof about 15 to about 25% cinnamon leaf oil or an active ingredient thereof; about 5 to about 15% tea tree oil or an active ingredient thereof; and about 15 to about 25% Eucalyptus globulus organic oil or an active ingredient thereof.

In one embodiment of the first aspect, the composition comprises: about 20% bergamot essential oil or an active ingredient thereof; about 16% clove bud oil or an active ingredient thereof; about 16% cinnamon leaf oil or an active ingredient thereof; about 12% tea tree oil or an active ingredient thereof; about 20% Eucalyptus globulus organic oil or an active ingredient thereof; and about 16% Trichosanthes fruit extract or an active ingredient thereof.

In one embodiment of the first aspect, the therapeutic composition comprises an oral dosage form. In one embodiment of the first aspect, the oral dosage form comprises a lozenge or a lollipop. In one embodiment of the first aspect, the oral dosage form further comprises about 0.5% to about 1% cinnamon leaf oil and 0.25% to about 0.5% Trichosanthes fruit extract. In another embodiment of the first aspect, the oral dosage form comprises one or more of water, sugar, corn syrup, and natural or artificial flavorings.

In a second aspect, the disclosure provides a method of treating a respiratory ailment associated with CRS in a subject in need thereof, comprising: a) administering a therapeutically effective amount of a composition to the subject, the composition comprising: i) a therapeutically effective amount of a Trichosanthes fruit extract or active ingredient thereof, and ii) a therapeutically effective amount of a second essential oil or active ingredient thereof; and b) reducing one or more symptoms of CRS and the respiratory ailment in the subject.

In one embodiment of the second aspect, the method further includes increasing fev1 in the subject about 7%-35%. In one embodiment of the second aspect, the increase in fev1 is about 12%. In one embodiment of the second aspect, the increase in fev1 is about 15%. In one embodiment of the second aspect, the respiratory ailment is asthma or chronic obstructive pulmonary disorder (COPD). In one embodiment of the second aspect, the composition is administered to the subject via oral, nasal, topical, buccal, sublingual, inhalation, insufflation, or transmucosal administration. In one embodiment of the second aspect, the composition is administered once, twice, or three times daily. In one embodiment of the second aspect, the respiratory ailment is associated with Mycoplasma or Chlamydia infection. In one embodiment of the second aspect, the method further includes monitoring a titer of Mycoplasma or Chlamydia in the subject.

In one embodiment according to any of the preceding aspects or embodiments thereof, the method further includes increasing an asthma assessment score in the subject by a value of about 1 to about 10. In one embodiment, the increase in the asthma assessment score is about 5.

In a third aspect, the disclosure provides a dispensing device, comprising: a therapeutically effective amount of a therapeutic composition, the therapeutic composition comprising i) a therapeutically effective amount of a Trichosanthes fruit extract or one or more active ingredients thereof, and ii) optionally, a second essential oil or active ingredient thereof, wherein the device is configured to dispense the therapeutic composition to a subject either nasally or orally.

In one embodiment of the third aspect, the device further comprises an absorbant material comprising the therapeutic composition. In one embodiment of the third aspect, the device is a nebulizer, an insufflation device, a syringe, an intranasal spray device, a pump sprayer, an inhaler, an intranasal inhaler, or a personal protection device. In one embodiment of the third aspect, the nebulizer is a compressor nebulizer, an ultrasonic nebulizer. In one embodiment of the third aspect, the inhaler is an intranasal inhaler. In one embodiment of the third aspect, the personal protection device is an N95 respirator, a surgical mask, a dust mask, a face mask, or a filtered breathing protection device. In one embodiment of the third aspect, the filtered breathing protection device comprises a replaceable vapor cartridge, a particulate filter, or an insert.

In a fourth aspect, the disclosure provides a kit, comprising a dispensing device according to the third aspect and any embodiments thereof.

In a fifth aspect, the disclosure provides a method of treating chronic rhinosinusitis (CRS) in a subject in need thereof including a) administering a therapeutically effective amount of a composition to the subject, the composition comprising therapeutically effective amounts of i) a bergamot essential oil, ii) a clove bud essential oil, iii) a cinnamon leaf essential oil, iv) a star anise essential oil, v) a tea tree essential oil, and vi) optionally, caffeine; and b) reducing one or more symptoms of CRS in the subject.

In one embodiment of the fifth aspect, the composition comprises an oral dosage form.

In another embodiment of the fifth aspect, the oral dosage form comprises a lozenge or a lollipop.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF DRAWINGS

FIG. 1: Dispenser device for therapeutic composition. In FIG. 1, a contemplated dispenser device 10, such as an intranasal inhaler, is shown whereby a user can self-administer a therapeutic composition as described herein. The dispenser device 10 includes a reservoir 12 contained within a body 14 for holding a therapeutic composition 16. The dispenser device 10 further includes an extension 18 with a first orifice 20 at an upper end and a second orifice 22 at a lower end to allow air (indicated by dashed arrow) to pass over/through a wick 24 that extends from the reservoir 12 into the extension. The wick 24 can transport the therapeutic composition 16 from the reservoir 12 up through the extension 18 and out the first orifice 20, for example, when the user inhales. In this way, air carrying the therapeutic composition 16 can be inhaled by the user to dispense the composition into the nasal cavity, air passageways, and/or lungs of the user. The dispenser device 10 further includes a cover 26 to prevent evaporation of the therapeutic composition 16 when the device is not in use.

FIG. 2: Respiratory protective device for therapeutic composition. In FIG. 2, a personal protective device 100 is shown that is designed to be worn over a user's nose and mouth. The device 100 includes a filter material 102 and an attachment feature 104, for example, an elastic strap that holds the device to a user's face when worn. The device 100 includes a pocket 106 in which an insert 108 impregnated with a therapeutic composition is held (though other means, such as fasteners, adhesive, velcro, and the like can be used in lieu of or in addition to a pocket). When in use, a user wearing the device 100 breathes in causing air (indicated by dashed arrow) to pass through the material 102 and through and/or around the insert 108. In this way, air carrying a therapeutic composition held within the insert 108 (and/or within the filter material 102) is inhaled by the user to dispense an effective amount of the composition into the nasal cavity, air passageways, and lungs of the user.

### DETAILED DESCRIPTION

All publications, patents and patent applications cited herein are hereby expressly incorporated by reference in their entirety for all purposes.

Before describing the methods and compositions of the disclosure in detail, a number of terms will be defined. As used herein, the singular forms “a,” “an,” and “the” include plural referents unless the context clearly dictates otherwise. For example, reference to “a metabolite” means one or more metabolites.

It is noted that terms like “preferably,” “commonly,” and “typically” are not utilized herein to limit the scope of the methods and compositions as described herein or to imply that certain features are critical, essential, or even important to the structure or function of the claimed invention.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “respiratory ailment” refers to diseases such as bronchitis, asthma, and COPD.

As used herein, the term “viral infection” refers to an infection of an individual with one or more viruses, such as a coronavirus, influenza, Epstein-Barr virus, respiratory syncytial virus, dengue virus, zika virus, west Nile virus, variola virus, cytomegalovirus, and human immunodeficiency virus (HIV).

As used herein, the term “bacterial infection” refers to an infection of an individual with one or more bacteria including, for example, group A Streptococcus.

As used herein, the terms “therapeutic amount,” “therapeutically effective amount,” and “effective amount” can be used interchangeably and refer to an amount of a substance that becomes available through the appropriate route of administration to treat a patient for a disorder, a condition, or a disease.

As used herein, the terms “composition” and “formulation” can be used interchangeably and refer to single substances and/or combinations of substances that can be used for therapeutic purposes, as described herein.

As used herein, the terms “combination” and “blend” can be used interchangeably and refer to mixtures of substances that can be used for therapeutic purposes, as described herein.

As used herein, the terms “treating,” “treat,” or “treatment” refer to either preventing, providing symptomatic relief, or curing a patient's disorder, condition, or disease.

As used herein, the term “CRS symptom” refers to a symptom associated with or indicative of CRS. For example, a CRS symptom may be nasal congestion, respiratory congestion, sinusitis, headache, post-nasal drip, wheezing, sneezing, coughing, shortness of breath, insomnia, and mucus production in the nasal cavity and/or respiratory tract and/or lungs.

As used herein, the terms “patient” and/or “subject” and/or “individual” can be used interchangeably and refer to an animal. For example, the patient, subject, or individual can be a mammal, such as a human.

As used herein, the terms “disorder,” “condition,” or “disease” refer to respiratory ailments caused by or associated with, for example, asthma, rhinitis, sinusitis, chronic rhinosinusitis allergies, bronchitis, chronic obstructive pulmonary disease (COPD), bacterial infections (e.g., Chlamydia pneumoniae, Mycoplasma pneumoniae, Haemophilus influenzae, Streptococcus pneumoniae, pneumococcus, etc.), and/or viral infections (e.g., the common cold, influenza, etc.).

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

### Overview

Described herein are therapeutic compositions for controlling (e.g., reducing symptoms) and/or curing CRS and associated respiratory ailments. Further, the therapeutic compositions described herein can reduce the need for the use of standard medications for the treatment of CRS and/or respiratory ailments and thereby reduce the cost associated with treatment. The therapeutic compositions of the present disclosure include new formulations of essential oils that can reduce symptoms of CRS, restore respiratory function, and provide extended relief to patients treated therewith.

In some embodiments, the present disclosure is directed to therapeutic compositions that include active ingredients with antioxidant, antibacterial, antifungal, anti-inflammatory, antibiofilm, and/or antiviral properties.

In some embodiments, the present disclosure is directed to treatment of individuals with acute or chronic respiratory ailments by administration of therapeutic compositions.

In some embodiments, the therapeutic compositions contemplated herein are effective at combating bacterial infections and/or biofilms involving gram-positive (e.g., Bacillus subtilis, Bacillus cereus, and Staphylococcus aureus, etc.) and/or gram-negative (e.g., Escherichia coli, Klebsiella pneumoniae, Pseudomonas aeruginosa, Proteus vulgaris, etc.) bacteria. For example, administration of a contemplated therapeutic composition kills bacteria that form biofilms or reduces the formation of biofilms by bacteria.

### Therapeutic Compositions and Dosage Forms

Therapeutic compositions contemplated herein include one or more essential oils and/or active ingredients of essential oils disclosed herein. Examples of essential oils contemplated herein include those derived from lemongrass, lavender, cinnamon, Eucalyptus (e.g., Eucalyptus globulus, Eucalyptus camaldulensis), tea tree (Melaleuca alternifolia), clove, Citrus plants (e.g., Citrus x paradisi, Citrus limonum, Citrus bergamia (bergamot), Citrus aurantium, and others), Murraya siamensis, and Trichosanthes species.

Contemplated Trichosanthes species that can be used include one or more of Trichosanthes baviensis, Trichosanthes cochinchinensis, Trichosanthes cucumerina, Trichosanthes cucumerina var. anguina, Trichosanthes dioica, Trichosanthes dunniana, Trichosanthes fissibracteata, Trichosanthes globosa, Trichosanthes homophylla, Trichosanthes kerrii, Trichosanthes kinabaluensis, Trichosanthes kirilowii, Trichosanthes laceribractea, Trichosanthes lepiniana, Trichosanthes montana, Trichosanthes pedata, Trichosanthes pendula, Trichosanthes pilosa, Trichosanthes pentaphylla, Trichosanthes postarii, Trichosanthes quinquangulata, Trichosanthes reticulinervis, Trichosanthes rosthornii, Trichosanthes rubiflos, Trichosanthes rugatisemina, Trichosanthes schlechteri, Trichosanthes sericeifolia, Trichosanthes subrosea, Trichosanthes subvelutina, Trichosanthes tricuspidata, Trichosanthes truncata, Trichosanthes villosa, and Trichosanthes wallichiana.

### Essential Oil/Active Ingredient Characteristics:

Essential oils and/or active ingredients thereof contemplated for use in the therapeutic compositions described herein include those that have antibacterial, antifungal, antiviral, and/or antibiofilm qualities. These oils, when used in conjunction, combat CRS and related microbial and viral infections. Specific essential oils and active ingredients thereof contemplated for use herein include:

1. Cinnamon bark oil with active ingredients including cinnamaldehyde, eugenol, isoeugenol, carvacrol, linalool, phenols, peroxides, ketones, and guaiazulene. This essential oil had the greatest strength in breaking up biofilms and has been shown to reduce beta lactamase from E. coli. Moreover, cinnamon bark oil kills Mycoplasma and Chlamydia pneumocci and is effective against influenza.

2. Eucalyptus oil with active ingredients including 1,8 cineole, alpha pinene, and camphor.

3. Tea tree oil with active ingredients including terpinenes terpinen-4-ol, cymene, and d-limonene. This essential oil attacks the cell wall and membranes of both gram negative and gram positive bacteria, activates apoptosis, reduces cytokine production, inhibits cox2, inhibits histamine release, and permeates the liposomal system causing leakage of potassium.

4. Bergamot oil with active ingredients including flavonoids, naringin, hesperidin, and linalool. This essential oil includes linalyl acetate, which attacks the lipid component of the cytoplasmic membrane.

5. Clove oil with active ingredients including oleic acids, phenylpropanoids, beta caryophlene, eugenol, oleoresin, and flavones. This essential oil partitions lipids of the bacterial cell wall and mitochondria disrupting cell structures and rendering them permeable.

6. Trichosanthes fruit extract with active ingredients including vitamin A, Vitamin C, terpinene, linalool, tannins, menthol, borneol, flavonoids, glycosides, and phenolics.

7. Star anise essential oil (from Illicium verum) with major constituents of (E)-anethole, foeniculin, methyl chavicol, limonene, linalool, nerolidol, and cinnamyl acetate.

Additional compounds and/or active ingredients of Trichosanthes species contemplated for use herein include (e)-beta-ocimene, anethole, b-elemen, borneol, cadina-1,4-diene, cadinene isomer, gamma-cadinene, alpha-cadinene, delta-cadinene, camphor, beta-caryophyllene, alpha-cedrene, beta-cedrenea, cedrol crystals, alpha-chamigrene, alpha-copaene, ethyl amyl ketone, eucalyptol, farnesene, alpha-farnesene, trans beta-farnesene, gamma-terpinene, geranoxide, geranyl acetate, d-germacrene, guaiene, alpha-humulene, isoborneol, isobornyl acetate, isolongifolene, levo-juneol, linalool, menthol, menthone, menthyl acetate, methyl benzoate, methyl chavicol, methyl salicylate, muurolene gamma, alpha-muurolol, neryl acetate, nopyl acetate, oleyl alcohol, para cresyl methyl ether, para cymene, alpha-pinene, terpinyl acetate, tetradecane, thujopsadien, thujopsene, thymol, widdrol, and ylangene, and subsets or various combinations thereof.

Contemplated essential oils from Trichosanthes fruit extracts inhibit histamine release and nitric oxide production. Further, and importantly, this essential oil has anti-inflammatory effects. This essential oil also enhances activities of antioxidant enzymes such as superoxide dismutase and glutathione peroxidase.

Essential oils used in the therapeutic compositions disclosed herein, with the exception of bergamot (obtained by a cold pressing process of the fruit peel), can be extracted using steam distillation from fruit juices, leaves, branches, or roots.

Contemplated therapeutic formulations including specific combinations of the essential oils described above (and/or a subset of their active ingredients) directly attack bacterial infections by killing bacteria and also directly reduce associated inflammation by downregulating inflammatory responses in a treated subject. Therefore, the present therapeutic combinations combat CRS and associated respiratory ailments both at the site of infection and at a site of inflammation, which can be remote from the causative bacterial infection.

A distinct advantage that the contemplated therapeutic formulations have over standard of care pharmaceutical treatments, such as steroids, is that by administration directly into the nares, airways, and lungs of a treated individual, they directly treat extent infections and do not undergo first-pass metabolism. Moreover, by directly killing occult infections underlying CRS and respiratory ailments, the therapeutic formulations of the present disclosure are curative rather than simply symptom reducing, like standard of care treatments and other products available from Bayer, Vicks, Walgreens, and Poysan used for cosmetic application. Such products feature ingredients that can reduce or mask symptoms associated with CRS and/or respiratory ailments, but unlike the present therapeutic formulations, they are not effective at treating the underlying causes of CRS, asthma, COPD, and related respiratory ailments.

Amounts of an essential oil or an active ingredient of an essential oil contained within a therapeutic composition contemplated herein can be about 0.1%, 0.2%, 0.3%, 0.4%, 0.5%, 0.6%, 0.7%, 0.8%, 0.9%, 1%, 2%, 3%, 4%, 5%, 10%, 15%, 20%, 25%, 50%, or 100% of the therapeutic composition, as well as any specific percentage between about 0.0001% and 100%. Further, contemplated amounts of essential oils or active ingredients thereof can be about 0.01 to about 1%, or about 0.1 to about 2%, or about 0.1 to about 5%, or about 1 to about 10%, or about 2 to about 20%, or about 5 to about 50%, or about 10 to about 75%, or about 25 to about 75%, or about 1 to about 50%, about 5 to about 75%, about 10 to about 90%, or about 0.1 to about 99%. Further ranges of contemplated amounts of essential oils or active ingredients thereof can be about 5% to about 15%, or 10% to about 20%, or about 15 to about 25%.

In some embodiments, a contemplated therapeutic formulation of the present disclosure can include a plurality of essential oils or active ingredients thereof. For example, contemplated therapeutic compositions can be as described below in Table Nos. 1A-1E. In each instance in the tables below, an essential oil may be replaced by one or more active ingredients of an essential oil. In this way, essential oils and their active ingredients (or some subset thereof) can be considered as interchangeable unless otherwise indicated.

TABLE NO. 1A
Therapeutic Blend Formulation

| Component | Percent |
|---|---|
| First essential oil | 0.1 to 99.9% |
| Second essential oil | 0.1 to 99.9% |

TABLE NO. 1B
Therapeutic Blend Formulation

| Component | Percent |
|---|---|
| First essential oil | 0.1 to 33.3% |
| Second essential oil | 0.1 to 33.3% |
| Third essential oil | 0.1 to 33.3% |

TABLE NO. 1C
Therapeutic Blend Formulation

| Component | Percent |
|---|---|
| First essential oil | 0.1 to 25% |
| Second essential oil | 0.1 to 25% |
| Third essential oil | 0.1 to 25% |
| Fourth essential oil | 0.1 to 25% |

TABLE NO. 1D
Therapeutic Blend Formulation

| Component | Percent |
|---|---|
| First essential oil | 0.1 to 20% |
| Second essential oil | 0.1 to 20% |
| Third essential oil | 0.1 to 20% |
| Fourth essential oil | 0.1 to 20% |
| Fifth essential oil | 0.1 to 20% |

TABLE NO. 1E
Therapeutic Blend Formulation

| Component | Percent |
|---|---|
| First essential oil | about 0.1 to 16.7% |
| Second essential oil | about 0.1 to 16.7% |
| Third essential oil | about 0.1 to 16.7% |
| Fourth essential oil | about 0.1 to 16.7% |
| Fifth essential oil | about 0.1 to 16.7% |
| Sixth essential oil | about 0.1 to 16.7% |

In some embodiments, therapeutic compositions can have 1, 2, 3, 4, 5, 6, 7, 8, 9, 10, 11, 12, 13, 14, 15, or more essential oils or active ingredients thereof. Therapeutic compositions can also include a single essential oil, a single active ingredient, and one or more additives and/or carriers (e.g., solvents). Further, contemplated therapeutic compositions can also include only specific active ingredients from certain essential oils.

In specific embodiments, contemplated therapeutic compositions are described below in Table Nos. 2A-2D. In some embodiments, the percentages and amounts described in Table Nos. 2A-2D can be per unit dosage form.

TABLE NO. 2A
Therapeutic Blend Formulation

| Component | Percent |
|---|---|
| Bergamot essential oil | 20% |
| Clove bud oil | 16% |
| Cinnamon leaf oil | 16% |
| Eucalyptus globulus organic oil | 20% |
| Tea tree oil | 12% |
| Trichosanthes cucumerina fruit extract | 16% |

TABLE NO. 2B
Therapeutic Blend Formulation

| Component | Percent |
|---|---|
| Bergamot essential oil | about 15 to about 25% |
| Clove bud oil | about 15 to about 25% |
| Cinnamon leaf oil | about 15 to about 25% |
| Eucalyptus globulus organic oil | 20% |
| Tea tree oil | about 5 to about 15% |
| Trichosanthes cucumerina fruit extract | about 15 to about 25% |

TABLE NO. 2C
Therapeutic Blend Formulation

| Component | Percent |
|---|---|
| Bergamot essential oil | about 3 to 4% |
| Clove bud essential oil | about 40% |
| Cinnamon leaf essential oil | about 4% |
| Star Anise essential oil | about 10% |
| Tea tree essential oil | about 42% |
| Additional active ingredient | optionally, about 0.1 to about 0.3% |

TABLE NO. 2D
Therapeutic Blend Formulation

| Component | Amount |
|---|---|
| Bergamot essential oil | about 3 to 4 g |
| Clove bud essential oil | about 40 g |
| Cinnamon leaf essential oil | about 4 g |
| Star Anise essential oil | about 10 g |
| Tea tree essential oil | about 42 g |
| Caffeine | optionally about 50 to about 200 mg |

Therapeutic compositions can further include one or more excipients, and a ratio of a therapeutically effective amount of one or more essential oils and/or active ingredients to an amount of one or more excipients (weight:weight) can be about 1:0.3 to about 1:20 or about 1:1 to about 1:10. The excipients can include a diluent, a solubilizer, an alcohol, a binder, a controlled release polymer, a disintegrant, a colorant, a flavorant, a sweetener, an antioxidant, a preservative, a pigment, an additive, a filler, a suspension agent, or surfactant.

Therapeutic compositions can also include one or more additional active ingredients that further aid in the treatment of respiratory ailments and associated symptoms. For example, contemplated additional active ingredient include one or more mild stimulants and/or bronchodilators. In one particular example, a contemplated additional active ingredient is caffeine.

Amounts of additional active ingredients that can be contained within any therapeutic composition contemplated herein can be about 0.1%, 0.2%, 0.3%, 0.4%, 0.5%, 0.6%, 0.7%, 0.8%, 0.9%, 1%, 2%, 3%, 4%, 5%, or 10% of the therapeutic composition, as well as any specific percentage between about 0.0001% and 10%. Further, contemplated amounts of additional active ingredient can be about 0.01 to about 1%, or about 0.1 to about 2%, or about 0.1 to about 5%, or about 1 to about 10%.

In some embodiments, additional active ingredients can be included, for example, in an amount of about 1 to 10 mg, or about 10 to 50 mg, or about 50 to about 200 mg, or about 100 to 200 mg, or about 200 to 300 mg, or about 100 to 500 mg per unit dose, such as per oral dosage form. Specific contemplated examples of oral dosage forms include lozenges, lollipops, and the like.

Depending on the dosing regimen, each administration of a therapeutic composition including one or more essential oils and/or active ingredients can be performed, for example, by giving a patient a single unit dose (e.g., an inhalable or an oral dosage form), multiple unit doses, or continuously (e.g., via impregnated mask or device), or otherwise as disclosed herein. For example, a therapeutic composition can be formulated for oral, nasal, topical (including buccal and sublingual), inhalation, insufflation, or transmucosal administration. It is further contemplated that therapeutic compositions can be formulated for different routes of administration at the same time or within the same administration.

Compositions containing the active ingredients may also contain one or more inactive excipients such as diluents, lubricants, solubilizers, alcohols, binders, controlled release polymers, enteric polymers, disintegrants, colorants, flavorants, sweeteners, antioxidants, preservatives, pigments, additives, fillers, suspension agents, surfactants (for example, anionic, cationic, amphoteric and nonionic), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

### Candy

In some embodiments, a therapeutic composition can be administered in the form of an oral dosage form such as a candy, for example, a hard candy. Infection of the oral pharynx can be due to a virus, bacteria, fungus, or a biofilm. Having a candy that a patient sucks on and maintains in their mouth like a lollipop or lozenge is an efficacious way to orally deliver therapeutic compositions that have antifungal, antibacterial, antiviral, and antibiofilm activities for combatting CRS. However, a challenge when creating these candies was to rid the mixture of bitter taste associated with Trichosanthes fruit extract, which is not problematic when administered via the nares or inhaled where contact with taste buds is limited or completely avoided. The formulated candies of the present disclosure are pleasant tasting and have been accepted by 98% of patients when subjected to a taste test.

Contemplated candies for adults include two or more essential oils. For example, one candy can include about 1% cinnamon leaf and about 0.5% Trichosanthes fruit extract. Concentrations of essential oils can be varied based on a number of factors, as described herein elsewhere. In addition, candies contemplated for adults can include flavors typically more preferred by adults, such as a coffee flavor. In such instances, the candies can further include caffeine as an additional active ingredient. In some examples, specific flavors, such as coffee or espresso can be indicative of the presence of caffeine in the formulation. For example, candies for children can include about 0.5% cinnamon leaf and 0.25% Trichosanthes fruit extract. Additional ingredients can include flavorants, polysaccharides, colorants, and the like. For example, candies contemplated herein can include a blend of essential oils (active ingredients) and pectin, sugar, and honey (inactive ingredients).

Patients exhibiting common cold symptoms who were administered a candy experienced symptoms clearing in less than a day. Such oral dosage forms further provide a psychological benefit over hospital visits and taking steroids for respiratory ailments, as they do not have a medical connotation. Further, giving a therapeutic candy to a child is a far easier treatment plan than taking the child to an emergency room for a nebulizer treatment. It is believed that such treatment plans are the more efficacious and administration of the presently disclosed compositions avoid the medical complications of hospital drugs.

### Therapeutic Methods

Some aspects of the present disclosure are directed to methods of treating a respiratory ailment in a subject in need thereof. For example, a contemplated method includes administering a therapeutically effective amount of a therapeutic composition to the subject and reducing one or more symptoms of the respiratory ailment in the subject.

Examples of symptoms that can be reduced include, nasal and/or respiratory congestion, shortness of breath, mucus production in the nasal cavity and/or respiratory tract and/or lungs. Symptom reduction can be a result of one or more of decreased biofilm mass, decreased bacterial density, increased lung capacity, among others, that results from treatment with the therapeutic composition.

In another embodiment, a contemplated method includes administering a therapeutically effective amount of a therapeutic composition to a subject and decreasing the need for using the subject's standard treatment for the respiratory ailment. For example, the method can decrease the number of instances when the subject uses or feels the need to use a steroid against one or more symptoms of the respiratory ailment.

In one particular embodiment, a method of treating chronic rhinosinusitis (CRS) in a subject in need thereof includes a) administering a therapeutically effective amount of a composition to the subject, the composition including i) a therapeutically effective amount of a Trichosanthes fruit extract or one or more active ingredients thereof, and ii) optionally, a second essential oil or active ingredient thereof; and b) reducing one or more symptoms of CRS in the subject. The method can reduce one or more of nasal and/or respiratory congestion, post-nasal drip, wheezing, sneezing, coughing, shortness of breath, insomnia, mucus production in the nasal cavity and/or respiratory tract and/or lungs.

Contemplated methods can reduce one or more of nasal and/or respiratory congestion, post-nasal drip, wheezing, sneezing, coughing, shortness of breath, insomnia, mucus production in the nasal cavity and/or respiratory tract and/or lungs. In another embodiment, reduction in one or more symptoms can be a result of one or more of decreased biofilm mass or decreased bacterial density in the subject's respiratory tract and/or increased lung capacity. In another embodiment, contemplated methods can reduce inflammation within the respiratory tract of the subject.

In another embodiment, a method of treating a respiratory ailment associated with CRS in a subject in need thereof can include a) administering a therapeutically effective amount of a composition to the subject, the composition comprising: i) a therapeutically effective amount of a Trichosanthes fruit extract or active ingredient thereof, and ii) a therapeutically effective amount of a second essential oil or active ingredient thereof; and b) reducing one or more symptoms of CRS and the respiratory ailment in the subject.

Contemplated respiratory ailments that can be treated include asthma or chronic obstructive pulmonary disorder (COPD). Administration may be by oral, nasal, topical, buccal, sublingual, inhalation, insufflation, or transmucosal administration. In one embodiment, the composition can be administered once, twice, or three times daily. In one embodiment, the respiratory ailment is associated with Mycoplasma or Chlamydia infection. In one embodiment, the method includes monitoring a titer of Mycoplasma or Chlamydia in the subject. Monitoring titers of Mycoplasma or Chlamydia in a subject can be by conventional approaches, such as by measurement of IgM and IgG antibody concentrations in a patient's blood.

In some embodiments, administration of the contemplated formulations in the form of a hard candy, such as a lozenge or lollipop provides a therapeutic advantage over other routes of administration for treating respiratory ailments. For example, hard candy is particularly well suited for delivering the therapeutic composition sublingually (and instructions to the user to keep the candy under his or her tongue are contemplated herein to aid the user to maximize effectiveness and quicken response time for the therapeutic composition). Moreover, sublingual delivery provides a further advantage of routing active ingredients of the therapeutic compositions (e.g., essential oils and/or additional active ingredients) directly to the oral pharynx more quickly, which is particularly relevant for treating CRS and respiratory ailments.

In another embodiment, a method of treating a respiratory ailment associated with CRS in a subject in need thereof can include a) administering a therapeutically effective amount of a composition to the subject in a hard candy form, such as a lozenge or lollipop, the composition comprising: i) a formulation as described in any of Table Nos. 2A-2D and b) reducing one or more symptoms of CRS and the respiratory ailment in the subject.

Based on the disclosure herein, the person of ordinary skill in the art will select an appropriate dosage regimen for the administration of the therapeutic composition.

Contemplated doses can be, for example, about 1 mL, or about 2 mL, or about 5 mL, or about 10 mL of a liquid therapeutic composition per administration.

In certain embodiments of the methods as otherwise described herein, a therapeutic composition (or an essential oil or an active ingredient thereof within the composition) is provided and/or administered at a dosage in the range of about 1 mg to about 200 mg, for example, about 3 mg to about 50 mg, about 6 mg to about 50 mg, or about 10 mg to about 25 mg per administration. However, in certain embodiments, no more than about 300 mg, or no more than about 200 mg, no more than about 100 mg, no more than about 75 mg, no more than about 50 mg, no more than about 25 mg, no more than about 10 mg, no more than about 6 mg, or no more than about 3 mg, or no more than about 1 mg of an essential oil or an active ingredient thereof is administered to a subject.

In certain embodiments, a therapeutic composition (or an essential oil or an active ingredient thereof within the composition) is provided and/or administered at a dosage in the range of about 1 mg to about 1000 mg, e.g., in the range of about 5 mg to about 800 mg, or in the range of about 5 mg to about 500 mg, or in the range of about 5 mg to about 200 mg, or in the range of about 5 mg to about 100 mg, or in the range of about 5 mg to about 50 mg, or in the range of about 10 mg to about 1000 mg, or in the range of about 10 mg to about 800 mg, or in the range of about 10 mg to about 500 mg, or in the range of about 10 mg to about 200 mg, or in the range of about 10 mg to about 100 mg, or in the range of about 10 mg to about 50 mg, or in any 5 mg range therein, such as about 1 to about 5 mg or about 6 mg to about 10 mg, and so on.

In other embodiments, a therapeutic composition (or an essential oil or an active ingredient thereof within the composition) is provided and/or administered at a dosage in the range of about 1 mg/mL to about 10 mg/mL, or about 5 mg/mL to about 20 mg/mL, or about 10 mg/mL to about 50 mg/mL, or about 100 mg/mL to about 2000 mg/mL.

Therapeutic compositions can be administered once, twice (BID), three times (TID), or more per day.

For example, in one embodiment, all doses described herein can be provided at about 1%, 2%, 3%, 4%, or 5%, or 10%, or 20%, or 30%, or 40%, or 50% the disclosed amount (e.g., 500 mg TID can be 5 mg, 25 mg, 15 mg, 20 mg, 25 mg, 50 mg, 150 mg, 200 mg, or 250 mg TID), or 150%, 200%, 300%, 400%, or 500% the disclosed amount.

As a general matter, suitable dosage amounts and dosing regimens may be selected in accordance with a variety of factors, including one or more of the subject's particular viral infection, bacterial infection, CRS, asthma, bronchitis, or COPD severity, the genetic profile, age, health, sex, diet, and/or weight of the subject, the route of administration alone or in combination with pharmacological considerations including the activity, efficacy, bioavailability, pharmacokinetic, and toxicological profiles of the particular therapeutic composition employed. Therefore, the dosing regimen to be employed may vary widely and may necessarily deviate from the dosage regimens set forth herein.

In another embodiment, the duration of administration of the therapeutic composition can be determined by indication of treatment effectiveness. For example, the therapeutic composition can be administered for a period of time during which a decrease in a symptom is observed. For example, treatment (a single or repeated administration over time of a therapeutic composition) can continue until a decrease in nasal and/or respiratory congestion, shortness of breath, mucus production in the nasal cavity and/or respiratory tract and/or lungs, biofilm mass, bacterial density, or increased lung capacity is observed.

In another embodiment, administration of a therapeutic composition can continue until a patient's respiration levels return to normal. In another embodiment, administration of a therapeutic composition can continue until fev1 in the subject increases by about 7%-35%. In one embodiment, administration can continue until the increase in fev1 is about 12% or about 15%.

In another embodiment, administration of a therapeutic composition can continue until a patient's asthma assessment score increases by about 1 to about 10, as measured by the Asthma Control Test™. In one embodiment, administration can continue until the patient's asthma assessment score increases is about 5.

### Devices

Devices contemplated herein for administration of the therapeutic composition (“dispenser devices” or “dispensing devices”) include nebulizers (e.g., a compressor nebulizer or an ultrasonic nebulizer), an insufflation device, a syringe, an intranasal spray device, such as an LMA MAD nasal intranasal mucosal atomization device, a pump sprayer, a respirator, and additional devices. Generally, any device that includes a reservoir for containing a therapeutic composition and a spraying or distributing mechanism to transfer the therapeutic composition from the reservoir into the nasal cavity and/or mouth, throat, and/or lungs of a patient is contemplated.

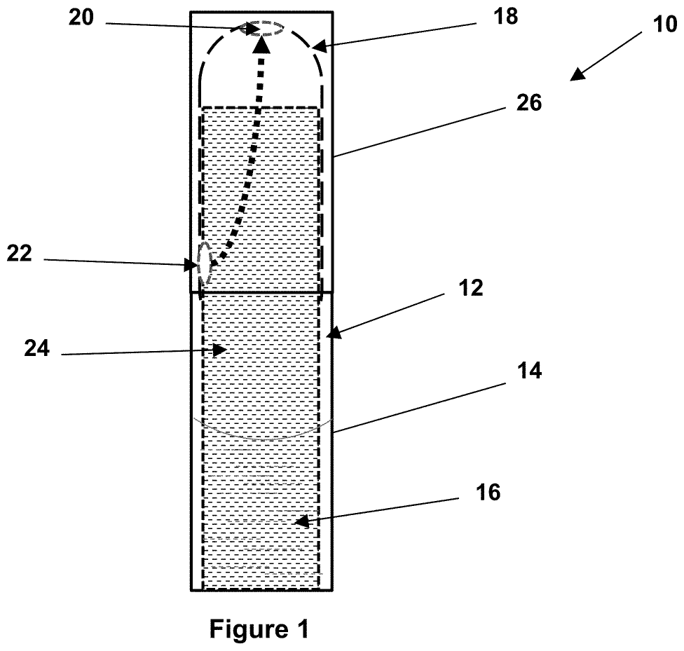

In one specific example, as shown in FIG. 1, a contemplated dispenser device 10, such as an intranasal inhaler, is shown whereby a user can self-administer a therapeutic composition as described herein. The device 10 includes a reservoir 12 (e.g., a 2 mL reservoir) contained within a body 14 for holding a therapeutic composition 16. The device 10 further includes an extension 18 with a first orifice 20 at an upper end and a second orifice 22 at a lower end to allow air (indicated by dashed arrow) to pass over and/or through an absorptive (e.g., cotton) wick 24 that extends from the reservoir into the extension and that contains the therapeutic composition 16 and travel up through the wick and/or extension and out the first orifice when the user inhales. In this way, air carrying the therapeutic composition 16 is inhaled by the user to dispense an effective amount of the composition into the nasal cavity, air passageways, and lungs of the user. The device 10 can further include a removable cover 26 to prevent evaporation of the therapeutic composition when not in use.

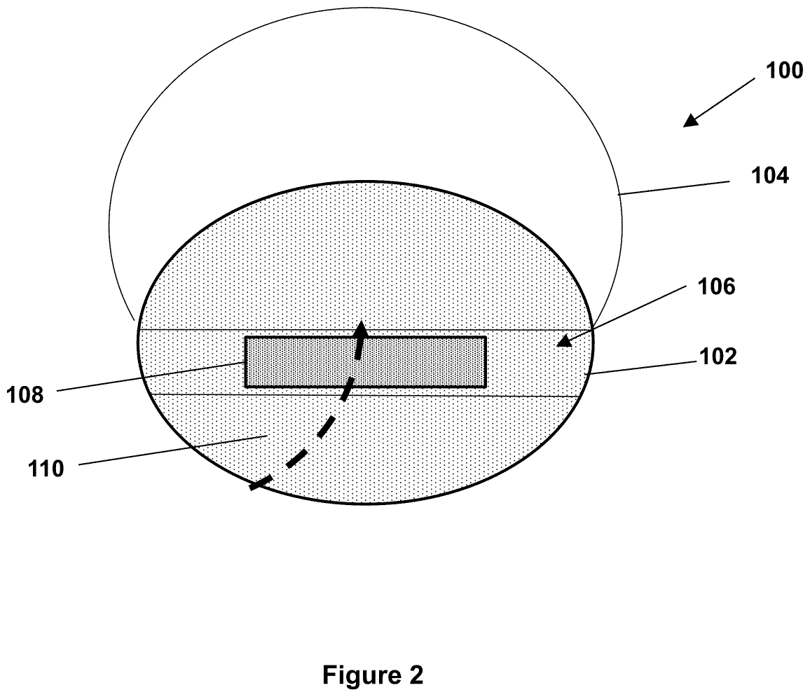

Another specific example of a dispenser device is shown in FIG. 2, which depicts a personal protective device 100 that is designed to be worn over a user's nose and mouth and can be used to protect the user from inhaling airborne particles. Examples of such devices include N95 respirators, surgical masks, dust masks, face masks, and any other filtered breathing protection devices. Such devices can include one or more filtering materials (e.g., the mask itself, or a replaceable vapor cartridge, particulate filter, and the like for use with a personal protective device, such as those available from 3M, Honeywell, Fightech, and others) that can be impregnated with a therapeutic composition of the present disclosure by spraying, dipping, or any other means. The device 100 includes a filter material 102 and an attachment feature 104, for example, one or more elastic straps, that holds the device to a user's face when worn. In the embodiment shown, the device 100 includes a pocket 106 in which an insert 108 impregnated with a therapeutic composition is held. Alternatively, the device 100 can include one or more vents (not shown), optionally containing a valve (not shown), into which the insert 108 can be removably secured. As a further embodiment, the insert 108 can be removeably secured to an interior surface of a personal protective device (e.g., near or around an opening for a vent). When in use, a user wearing the device 100 breathes in causing air (indicated by dashed arrow) to pass through the material 102 and through and/or around the insert 108. In this way, air carrying a therapeutic composition held within the insert 108 (and/or within the filter material 102) is inhaled by the user to dispense an effective amount of the composition into the nasal cavity, air passageways, and lungs of the user. Such devices 100 combine a physical barrier to airborne contaminants (i.e., a filter) with a pharmacological barrier (i.e., a therapeutic composition) to provide increased protection to users who may be exposed to particulates, allergens, and/or pathogens, such as bacteria and/or viruses.

Alternatively, it is envisioned that personal protection devices such as those described above can be configured for treatment/filtration of exhaled air.

In one specific embodiment, a contemplated therapeutic composition (e.g., an essential oil blend) can be applied to an insert 108 and made to fit an N95 mask. In this context, the insert 108 can have any shape to fit into or onto an interior surface of the mask, for example, a circular shape, and can have any appropriate size, such as about 0.5 inch to about 1.5 inches in diameter. The insert can also be any suitable material. In one specific embodiment, the insert is a bamboo paper or fabric. In a particular embodiment, a therapeutic composition is impregnated into a circular insert made of bamboo paper that has a diameter of 0.835 inches. Contemplated inserts will last from about 4 to about 7 days and can be replaced. In this way, a single N95 mask (or similar personal protection device) can be used for extended periods of time and the user can maintain the administration of a therapeutic composition being taken or change to a different composition carried in a different insert.

Additional devices such as “CPAP” machines used for sleep treating apnea may also be used to administer therapeutic compositions of the present disclosure.

Contemplated devices for dispensing the therapeutic compositions of the present disclosure can be intended for a single use or for repeated uses. In some embodiments, contemplated devices can be disposed of after use or refillable. In some embodiments, contemplated devices can be designed for about 1, about 10, or about 100 uses. For example, an insert 108 for a device can be replaced every 10 days. In some embodiments, contemplated devices can be used for about 1 week, or for about 1 month, or for about 6 months of daily use to provide an effective amount of a therapeutic composition to a user thereof. As used herein, the term daily use refers to administration of an effective amount of a therapeutic composition to an individual at least once per day. For example, daily use can be administration of an effective amount of a therapeutic composition to an individual once per day, or twice per day, or three times per day, or four to six times per day.

It is contemplated that concentrations of therapeutic compositions may be adjusted depending upon the device used for administration.

### Kits

As used herein, a kit may be a packaged collection of related materials, including, for example, a single package or a plurality of packages containing a single and/or a plurality of dosage forms and/or dispensing devices along with instructions for use.

In another embodiment, a kit includes a dispensing device and/or a solution, suspension, or other dosage form and instructions to use the device and/or apply the dosage form to the device or directly to the nasal cavity or respiratory tract as appropriate for the device and/or dosage form. Application can be by the device included in the kit or the dosage form, as described herein elsewhere.

The invention will be further described in the following examples, which do not limit the scope of the invention described in the claims.

### EXAMPLES

The Examples that follow are illustrative of specific embodiments of the invention, and various uses thereof. They are set forth for explanatory purposes only and are not taken as limiting the invention.

### Example No. 1: Treatment of Respiratory Ailments

### Introduction:

Disease of the nose and sinuses is the most common comorbidity associated with asthma. Rhinitis typically precedes the development of asthma. Rhinitis sinusitis and asthma may represent part of one disease process with manifestations at different sites. It is believed that by treating sinus infections, the symptoms of asthma and COPD can be controlled and/or cured. In the present study, the goal was to decrease airway inflammation in the sinuses, the oral airway, and in the lungs by treatment with an experimental therapeutic composition.

### Study Protocol

### 1.1 Patients and Treatments

A group of twenty four patients ranging in age from 17-64 years in age were treated for a period of 6 months with a therapeutic composition including an extract of Trichosanthes fruit. The extract included the components listed in Table No. 3.

TABLE NO. 3
Extract components

| Component | Percent |
|---|---|
| (E)-Beta-Ocimene | 0.026 |
| Anethole | 3.253 |
| B-Elemen | 0.503 |
| Borneol | 0.471 |
| Cadina-1,4-Diene | 0.407 |
| Cadinene isomer | 0.37 |
| Cadinene Gamma- | 1.588 |
| Cadinene, Alpha- | 0.599 |
| Cadinene, Delta | 7.298 |
| Camphor | 1.773 |
| Caryophyllene Beta | 10.389 |
| Cedrene Alpha | 1.107 |
| Cedrene Beta | 1.214 |
| Cedrol Crystals | 0.555 |
| Chamigrene, Alpha- | 0.148 |
| Copaene Alpha | 1.23 |
| Ethyl Amyl Ketone | 0.099 |
| Eucalyptol | 4.959 |
| Famesene | 0.166 |
| Farnesene Alpha | 11.039 |
| Farnesene, Trans Beta- | 0.56 |
| Gamma-Terpinene | 0.029 |
| Geranoxide | 0.059 |
| Geranyl Acetate | 1.017 |
| Germacrene D | 6.189 |
| Guaiene | 0.78 |
| Humulene Alpha - | 3.225 |
| Isoborneol | 0.397 |
| Isobornyl Acetate | 0.122 |
| Isolongifolene | 0.272 |
| Juneol, Levo- | 0.357 |
| Linalool | 0.427 |
| Menthol | 17.071 |
| Menthone | 0.28 |
| Menthyl Acetate | 1.537 |
| Methyl Benzoate | 0.061 |
| Methyl Chavicol | 0.163 |
| Methyl Salicylate | 0.289 |
| Muurolene Gamma | 1.165 |
| Muurolol Alpha- | 0.222 |
| Neryl Acetate | 0.101 |
| Nopyl Acetate | 0.348 |
| Oleyl Alcohol | 1.011 |
| Para Cresyl Methyl Ether | 0.17 |
| Para Cymene | 0.072 |
| Pinene Alpha | 0.06 |
| Terpinyl Acetate | 0.078 |
| Tetradecane | 0.08 |
| Thujopsadien | 0.244 |
| Thujopsene | 2.362 |
| Thymol | 0.076 |
| Widdrol | 0.103 |
| Ylangene | 0.308 |

The patients were administered about 1.5 mL in each nares of the therapeutic composition via 3 mL syringe. Treatment was repeated on a monthly basis until the patient obtained total relief. In most instances, only three treatments were necessary.

### 1.2 Patient Measurements

To assess treatment efficacy, patient spirometry was measured via incentive spirometer before treatment and 6 months after initiation of treatment with the therapeutic composition.

Further, patients were administered an asthma symptom assessment, such as the Asthma Control Test™ in which patients are assessed based on a series of questions with a numerical response that reflects the severity for particular assessment criteria related to asthma symptoms. Results were added to provide an overall score, which is indicative of the patient's overall health relating to asthma symptoms. Patients with scores of 20 and above have well controlled asthma symptoms, patients with scores of 16-19 have poor to inadequate control of asthma symptoms, and patients with scores of 15 and below have very poorly controlled asthma symptoms. The criteria assessed are presented in Table No. 4.

TABLE NO. 4
Asthma Symptom Assessment Criteria

1. In the past 4 weeks, how much of the time did your asthma keep you from getting as
much done at work, school or at home?

| | | | | | |
|---|---|---|---|---|---|
| All of the | Most of the | Some of the | A little of the | None of the | Total = |
| time (1) | time (2) | time (3) | time (4) | time (5) | |

2. During the past 4 weeks, how often have you had shortness of breath?

| | | | | | |
|---|---|---|---|---|---|
| More than | Once a | 3 to 6 | Once or | Not at | Total = |
| once a | day (2) | times per | twice a | all (5) | |
| day (1) | | week (3) | week (4) | | |

3. During the past 4 weeks, how often did your asthma symptoms (wheezing, coughing,
shortness of breath, chest tightness or pain) wake you up at night or earlier than usual in
the morning?

| | | | | | |
|---|---|---|---|---|---|
| 4 or more | 2 to 3 | Once a | Once or | Not at | Total = |
| nights a | nights per | week (3) | twice (4) | all (5) | |
| week (1) | week (2) | | | | |

4. During the past 4 weeks, how often have you used a rescue inhaler or nebulizer
medication (such as albuterol)?

| | | | | | |
|---|---|---|---|---|---|
| 3 or more | 1 to 2 | 2 to 3 | Once a | Not at | Total = |
| times per | times per | times per | week or | all (5) | |
| day (1) | day (2) | week (3) | less (4) | | |

5. How would you rate your asthma control during the past 4 weeks?

| | | | | | |
|---|---|---|---|---|---|
| Not | Poorly | Somewhat | Well | Completely | Total = |
| controlled | controlled | controlled | controlled | controlled | |
| at all (1) | (2) | (3) | (4) | (5) | |
| | | | | | Total = |

### Results:

As shown in Table Nos. 5 and 6, administration of the therapeutic composition was effective in restoring lung capacity and reducing asthma symptoms in all patients treated.

TABLE NO. 5
Patient Results - Spirometry.

| | | | | | | % normal | Spirometry | % normal | 6 Month | % | |
|---|---|---|---|---|---|---|---|---|---|---|---|
| | | | Height | Spirometry | Spirometry | spirometry | after | spirometry | change in | change in | |
| Patient | Sex | Age | (cm) | normal | pretreatment | pre-treatment | treatment | post-treatment | Spirometry | spirometry | saO₂ |
| A | M | 17 | 165 | 4080 | 3050 | 74.75 | 3600 | 88.24 | 550 | 13.48 | 97/99 |
| B | F | 20 | 160 | 3220 | 2600 | 80.75 | 3100 | 96.27 | 500 | 15.53 | 98/98 |
| C | M | 61 | 158 | 2400 | 1750 | 72.92 | 2100 | 87.50 | 350 | 14.58 | 94/96 |
| D | M | 24 | 170 | 4120 | 3750 | 91.02 | 4050 | 98.30 | 300 | 7.28 | 99/99 |
| E | M | 62 | 163 | 2720 | 2300 | 84.56 | 2550 | 93.75 | 250 | 9.19 | 98/98 |
| F | M | 32 | 168 | 3810 | 3050 | 80.05 | 3550 | 93.18 | 500 | 13.12 | 98/98 |
| G | F | 52 | 163 | 2540 | 2050 | 80.71 | 2400 | 94.49 | 350 | 13.78 | 96/98 |
| H | F | 25 | 158 | 2500 | 1800 | 72.00 | 2300 | 92.00 | 500 | 20.00 | 95/99 |
| I | M | 44 | 163 | 2700 | 2050 | 75.93 | 2350 | 87.04 | 300 | 11.11 | 98/98 |
| J | M | 56 | 173 | 3330 | 3050 | 91.59 | 3350 | 100.60 | 300 | 9.01 | 99/99 |
| K | F | 62 | 160 | 2170 | 1850 | 85.25 | 2150 | 99.08 | 300 | 13.82 | 95/97 |
| L | F | 52 | 163 | 2300 | 1600 | 69.57 | 2400 | 104.35 | 800 | 34.78 | 93/96 |
| M | M | 64 | 165 | 2750 | 2200 | 80.00 | 2650 | 96.36 | 450 | 16.36 | 98/98 |
| N | F | 28 | 152 | 2700 | 2150 | 79.63 | 2500 | 92.59 | 350 | 12.96 | 97/98 |
| O | F | 58 | 168 | 2470 | 2050 | 83.00 | 2350 | 95.14 | 300 | 12.15 | 96/98 |
| P | M | 33 | 165 | 3070 | 2500 | 81.43 | 2950 | 96.09 | 450 | 14.66 | 98/99 |
| Q | F | 36 | 168 | 3140 | 2700 | 85.99 | 3150 | 100.32 | 450 | 14.33 | 99/100 |
| R | M | 25 | 170 | 4120 | 3500 | 84.95 | 4000 | 97.09 | 500 | 12.14 | 99/99 |
| S | F | 48 | 157 | 2400 | 2000 | 83.33 | 2350 | 97.92 | 350 | 14.58 | 98/99 |
| T | M | 38 | 157 | 3160 | 2650 | 83.86 | 3150 | 99.68 | 500 | 15.82 | 98/99 |
| U | M | 31 | 168 | 2950 | 2400 | 81.36 | 2900 | 98.31 | 500 | 16.95 | 98/99 |
| V | M | 50 | 160 | 2940 | 2500 | 85.03 | 2900 | 98.64 | 400 | 13.61 | 97/99 |
| W | F | 43 | 155 | 2470 | 2150 | 87.04 | 2450 | 99.19 | 300 | 12.15 | 98/98 |
| X | M | 42 | 175 | 4110 | 3450 | 83.94 | 4100 | 99.76 | 650 | 15.82 | 100/100 |

| | | | |
|---|---|---|---|
| Average values | 425 | 14.47 | — |

All patient objective values improved over the course of a month and remarkable increases in percent spirometry were seen after the 6 month treatment period. In the American medical system if you are to get a 12% increase in fev1 (the amount of air a person can force from her lungs in one second), it is considered a therapeutic success. In the patients treated, the average percent increase in fev1 was 14.47%, and only 4 patients had increased fev1 of less than 12%.

TABLE NO. 6
Patient Results - Asthma Symptom Assessment.

| | Asthma Assessment | Asthma Assessment | Improvement in |
|---|---|---|---|
| | Score after | Score after | Asthma Assessment |
| Patient | treatment | treatment | Score |
| A | 17 | 22 | 5 |
| B | 16 | 22 | 6 |
| C | 15 | 20 | 5 |
| D | 18 | 23 | 5 |
| E | 18 | 24 | 6 |
| F | 15 | 23 | 8 |
| G | 13 | 22 | 9 |
| H | 17 | 21 | 4 |
| I | 16 | 22 | 6 |
| J | 19 | 24 | 5 |
| K | 13 | 20 | 7 |
| L | 14 | 19 | 5 |
| M | 20 | 24 | 4 |
| N | 19 | 23 | 4 |
| O | 16 | 20 | 4 |
| P | 18 | 23 | 5 |
| Q | 17 | 22 | 5 |
| R | 18 | 25 | 7 |
| S | 18 | 22 | 4 |
| T | 19 | 24 | 5 |
| U | 15 | 22 | 7 |
| V | 18 | 24 | 6 |
| W | 18 | 25 | 7 |
| X | 17 | 24 | 7 |

| | |
|---|---|
| Average value | 5.7 |

All patient subjective, self-assessed values improved during the treatment period. As a group, the patients experienced an average increase in asthma assessment score of 5.7. Only one patient had a score below 20 after treatment (19). These results demonstrate that the treatment regimen provided considerable improvement in asthma symptoms in all treated patients, with overall well-controlled asthma symptoms.

In addition, specific improvements in CRS symptoms were reported including decreased post-nasal drip, decreased wheezing, sneezing, and coughing, decreased insomnia (improved sleep), easier breathing, and fewer complications experienced with standard of care medications (e.g., steroids) because patients were able to reduce of stop taking such medications (e.g., steroids). All the patients improved with most if not all of their symptoms abated. Patients reported missing fewer days at work and school after receiving the treatments.

Several patients needed to get more than the standard 3 treatments, but all patients ultimately responded well to the treatments. Nighttime symptoms were reported by patients to be drastically reduced. Episodes of shortness of breath were either completely eliminated or diminished.

All the patients were able to stop routine use of steroid inhalers. Though some patients were still maintained on a lower dose of steroids, many were able to shed completely their dependence on oral steroids. The results of the asthma control test showed that only a small number of patients still needed their rescue inhalers. And, those patients that did were less reliant on them. Therefore, the present therapeutic composition is also effective against tachyphylaxis.

### Conclusion:

The Allergic March is a term used to describe the progression of allergic disease from the nose and sinuses down to the airways of the lung. As chronic rhinosinusitis (CRS) is a forerunner of asthma, it is believed that intervening with CRS may afford an opportunity to prevent the development of asthma or treat asthma in an individual. The inflammatory processes in the upper and lower airways share many common features. For example, severity of the disease in the upper airway parallels that in the lower airways. In addition, the same lymphocyte and eosinophil inflammation observed in the lower airways is seen in the upper airways. Similarly, the same mediators of disease, mast cells, lymphoid cells, interleukin, eotaxins, basophils, and cytokine expression, are also observed.

Presently there are no good treatments for CRS. Surgery for polyp removal is not a long-term solution. Steroids are often used but rarely cure the disease process and often makes it worse. Using multiple antibiotics to treat CRS also rarely works and can lead to antibiotic resistant microbes. The present study establishes that administration of a therapeutic composition containing an essential oil or specific active ingredients thereof provides profound and sustained relief of symptoms to patients suffering from CRS and the associated respiratory ailment, asthma. Treatment with therapeutic compositions including extracts of Trichosanthes fruit and other essential oils with antifungal, antibacterial, antiviral, and/or antibiofilm activities offer a new and natural alternative for combatting CRS and preventing or reducing development of associated respiratory ailments.

### Example No. 2: Improved Strategies for Treating Respiratory Ailments

### Introduction:

When treating young children for asthma, cooperation of the child is essential and the vehicle of administration for a medicine can be critical to obtain compliance and maximize efficacy. In the present example, a therapeutic composition was formulated as lollipops and hard candy in an experiment to determine whether children would be more compliant with taking the medicine and whether the dosage form (e.g., a lollipop) could promote keeping the formulation in the oral pharynx for an extended period of time.

### Study Protocol

### 1.1 Patients and Treatments

Twenty four children (aged 4-12) suffering from CRS symptoms were treated with 1 lollipop every eight hours for 2-3 days and symptoms were monitored during the treatment period. The formulation of the lollipops is described in Table No. 2C. No additional active ingredient was included in the formulation.

### Results:

All children were agreeable to taking the formulation in candy form, which was preferred over having to take the medications through pills, nebulizers, liquid administration, or inhalers. Of the 24 children that were treated, the CRS symptoms of all but two clinically improved over the course of treatment. Their collective symptoms included wheezing, cough, running nose, sinusitis, fatigue, and shortness of breath. Of the 22 responders, their symptoms resolved without needing to go to an emergency room or use a rescue inhaler. One patient needed emergent care in a hospital setting, and one patient received treatment with a ventolin inhaler.

### Conclusion:

These results establish the therapeutic effectiveness of the administered formulation in reducing and/or resolving CRS symptoms in children. Moreover, the route of administration of the essential oil formulation administered in the form of sucking candy improved treatment compliance in children and aided in the therapeutic activity of the essential oils by prolonging exposure of the oral pharynx to the formulation (by sucking the candy), as well as, by sublingual absorption of the essential oils.

Therefore, a child to be treated as described in this example will benefit by: a) the therapeutic effectiveness of the administered antimicrobial essential oil formulation; b) an improved treatment experience of receiving the formulation as a hardy candy rather than as an unpleasant nebulizer or inhaler treatment improving treatment compliance; and c) an improved treatment efficacy by a prolonged direct exposure of the upper respiratory system and oral pharynx to the therapeutic formulation by sucking which promotes sublingual absorption of the formulation and a faster and higher systemic concentration of the formulation.

Having described the invention in detail and by reference to specific aspects and/or embodiments thereof, it will be apparent that modifications and variations are possible without departing from the scope of the invention defined in the appended claims. More specifically, although some aspects of the present invention may be identified herein as particularly advantageous, it is contemplated that the present invention is not limited to these particular aspects of the invention. In some embodiments, values disclosed herein may alternatively vary in amount by ±10, 20, or 30% from values disclosed and remain within the scope of the contemplated invention. All patents and publications are incorporated herein by reference in their entireties.

What is claimed is:

1. A method of treating chronic rhinosinusitis (CRS) in a subject in need thereof, comprising:
a) administering a therapeutically effective amount of a composition to the subject, the composition comprising:
i) a therapeutically effective amount of a Trichosanthes fruit extract or one or more active ingredients thereof, and
ii) optionally, a second essential oil or active ingredient thereof; and
b) reducing one or more symptoms of CRS in the subject.

2. The method of claim 1, wherein the method reduces one or more of nasal and/or respiratory congestion, post-nasal drip, wheezing, sneezing, coughing, shortness of breath, insomnia, mucus production in the nasal cavity and/or respiratory tract and/or lungs.

3. The method of claim 1, wherein reduction in one or more symptoms is a result of one or more of decreased biofilm mass or decreased bacterial density in the subject's respiratory tract and/or increased lung capacity.

4. The method of claim 1, wherein the method reduces inflammation within the respiratory tract of the subject.

5. The method of claim 1, wherein the composition comprises about 0.1 to about 99% of the Trichosanthes fruit extract or an active ingredient thereof.

6. The method of claim 5, wherein the active ingredient of Trichosanthes fruit extract comprises one or more of (e)-beta-ocimene, anethole, b-elemen, borneol, cadina-1,4-diene, cadinene isomer, gamma-cadinene, alpha-cadinene, delta-cadinene, camphor, beta-caryophyllene, alpha-cedrene, beta-cedrenea, cedrol crystals, alpha-chamigrene, alpha-copaene, ethyl amyl ketone, eucalyptol, farnesene, alpha-farnesene, trans beta-farnesene, gamma-terpinene, geranoxide, geranyl acetate, d-germacrene, guaiene, alpha-humulene, isoborneol, isobornyl acetate, isolongifolene, levo-juneol, linalool, menthol, menthone, menthyl acetate, methyl benzoate, methyl chavicol, methyl salicylate, muurolene gamma, alpha-muurolol, neryl acetate, nopyl acetate, oleyl alcohol, para cresyl methyl ether, para cymene, alpha-pinene, terpinyl acetate, tetradecane, thujopsadien, thujopsene, thymol, widdrol, and ylangene, and combinations thereof.

7. The method of claim 6, wherein the active ingredient of Trichosanthes fruit extract comprises at least 4 or more of (e)-beta-ocimene, anethole, b-elemen, borneol, cadina-1,4-diene, cadinene isomer, gamma-cadinene, alpha-cadinene, delta-cadinene, camphor, beta-caryophyllene, alpha-cedrene, beta-cedrenea, cedrol crystals, alpha-chamigrene, alpha-copaene, ethyl amyl ketone, eucalyptol, farnesene, alpha-farnesene, trans beta-farnesene, gamma-terpinene, geranoxide, geranyl acetate, d-germacrene, guaiene, alpha-humulene, isoborneol, isobornyl acetate, isolongifolene, levo-juneol, linalool, menthol, menthone, menthyl acetate, methyl benzoate, methyl chavicol, methyl salicylate, muurolene gamma, alpha-muurolol, neryl acetate, nopyl acetate, oleyl alcohol, para cresyl methyl ether, para cymene, alpha-pinene, terpinyl acetate, tetradecane, thujopsadien, thujopsene, thymol, widdrol, and ylangene.

8. The method of claim 5, wherein the composition comprises:
about 10% to about 20% Trichosanthes fruit extract or an active ingredient thereof.

9. The method of claim 8, wherein the composition further comprises:
about 15 to about 25% bergamot essential oil or an active ingredient thereof;
about 15 to about 25% clove bud oil or an active ingredient thereof;
about 15 to about 25% cinnamon leaf oil or an active ingredient thereof;
about 5 to about 15% tea tree oil or an active ingredient thereof; and
about 15 to about 25% Eucalyptus globulus organic oil or an active ingredient thereof.

10. The method of claim 9, wherein the composition comprises:
about 20% bergamot essential oil or an active ingredient thereof;
about 16% clove bud oil or an active ingredient thereof;
about 16% cinnamon leaf oil or an active ingredient thereof;
about 12% tea tree oil or an active ingredient thereof;
about 20% Eucalyptus globulus organic oil or an active ingredient thereof; and
about 16% Trichosanthes fruit extract or an active ingredient thereof.

11. The method of claim 1, wherein the composition comprises an oral dosage form.

12. The method of claim 11, wherein the oral dosage form comprises a lozenge or a lollipop.

13. The method of claim 11 or 12, wherein the oral dosage form further comprises about 0.5% to about 1% cinnamon leaf oil and 0.25% to about 0.5% Trichosanthes fruit extract.

14. The method of claim 11, wherein the oral dosage form comprises one or more of water, sugar, corn syrup, and natural or artificial flavorings.

15. A method of treating a respiratory ailment associated with CRS in a subject in need thereof, comprising:
a) administering a therapeutically effective amount of a composition to the subject, the composition comprising:
i) a therapeutically effective amount of a Trichosanthes fruit extract or active ingredient thereof, and
ii) a therapeutically effective amount of a second essential oil or active ingredient thereof; and
b) reducing one or more symptoms of CRS and the respiratory ailment in the subject.

16. The method of claim 15 further comprising increasing fev1 in the subject about 7%-35%.

17. The method of claim 16, wherein the increase in fev1 is about 12%.

18. The method of claim 16, wherein the increase in fev1 is about 15%.

19. The method of claim 15, wherein the respiratory ailment is asthma or chronic obstructive pulmonary disorder (COPD).

20. The method of claim 15, wherein the composition is administered to the subject via oral, nasal, topical, buccal, sublingual, inhalation, insufflation, or transmucosal administration.

21. The method of claim 20, wherein the composition is administered once, twice, or three times daily.

22. The method of claim 15, wherein the respiratory ailment is associated with Mycoplasma or Chlamydia infection.

23. The method of claim 22 further comprising monitoring a titer of Mycoplasma or Chlamydia in the subject.

24. The method of any of the preceding claims further comprising increasing an asthma assessment score in the subject by a value of about 1 to about 10.

25. The method of claim 24, wherein the increase in the asthma assessment score is about 5.

26. A dispensing device, comprising:
a therapeutically effective amount of a composition, the composition comprising
i) a therapeutically effective amount of a Trichosanthes fruit extract or one or more active ingredients thereof, and
ii) optionally, a second essential oil or active ingredient thereof,
wherein the device is configured to dispense the composition to a subject either nasally or orally.

27. The device of claim 26, wherein the device further comprises an absorbant material comprising the composition.

28. The device of claim 26 or 27, wherein the device is a nebulizer, an insufflation device, a syringe, an intranasal spray device, a pump sprayer, an inhaler, an intranasal inhaler, or a personal protection device.

29. The device of claim 28, wherein the nebulizer is a compressor nebulizer, an ultrasonic nebulizer.

30. The device of claim 28, wherein the inhaler is an intranasal inhaler.

31. The device of claim 28, wherein the personal protection device is an N95 respirator, a surgical mask, a dust mask, a face mask, or a filtered breathing protection device.

32. The device of claim 31, wherein the filtered breathing protection device comprises a replaceable vapor cartridge, a particulate filter, or an insert.

33. A kit, comprising a dispensing device according to any of claims 26-32.

34. A method of treating chronic rhinosinusitis (CRS) in a subject in need thereof, comprising:
a) administering a therapeutically effective amount of a composition to the subject, the composition comprising therapeutically effective amounts of
i) a bergamot essential oil,
ii) a clove bud essential oil,
iii) a cinnamon leaf essential oil
iv) a star anise essential oil,
v) a tea tree essential oil, and
vi) optionally, caffeine; and
b) reducing one or more symptoms of CRS in the subject.

35. The method of claim 34, wherein the composition comprises an oral dosage form.

36. The method of claim 35, wherein the oral dosage form comprises a lozenge or a lollipop.
